(19) United States
(12) Patent Application Publication
(10) Pub. No.: US 2020/0261662 A1
(43) Pub. Date: Aug. 20, 2020

(54) CANNULA FIXATION DEVICE

(71) Applicant: TheraCord LLC, Baltimore, MD (US)

(72) Inventor: Christopher CHIANG, Baltimore, MD (US)

(21) Appl. No.: 16/747,927

(22) Filed: Jan. 21, 2020

Related U.S. Application Data
(63) Continuation of application No. PCT/US2018/043056, filed on Jul. 20, 2018.
(60) Provisional application No. 62/535,491, filed on Jul. 21, 2017.

(51) Int. Cl. A61M 5/34; A61M 5/14; A61M 25/06; A61M 25/00

(52) U.S. Cl. CPC A61M 5/343; A61M 5/14; A61M 2210/1466; A61M 25/0014; A61M 2210/1458; A61M 25/06

(57) ABSTRACT

An embodiment in accordance with the present invention is directed to a device for fixation, stabilization, and securement of an intravascular cannula to tissue. The fixation device affixes relative to the cannula and cannulated tissue, allowing the needle to be inserted to a shallow depth and then rapidly secured with one motion, using just one hand. The device works to secure cannulae not only in small vessels but also in situations non-permissive to current state-of-the-art cannulas—such as on wet, adhesive-incompatible surfaces, in intra-operative applications, or when hands-free securement is required. The device can also be utilized to access the umbilical arteries and vein for purposes of placental perfusion for cord blood collection or to facilitate and extend the duration of conventional cord blood collection.

### FIELD OF THE INVENTION

The present invention relates generally to medical devices. More particularly, the present invention relates to a device for cannula fixation.

### BACKGROUND OF THE INVENTION

Umbilical cord blood is a source of transplantable Hematopoietic Stem Cells (HSC) unhindered by ethical issues or complex surgical extraction. Cord blood (CB) is collected from the umbilical cord and placenta after birth. Cord blood transplants are used in the established treatments of over 80 diseases, similar to bone marrow transplant, but with the advantage of having increased flexibility in immunological (e.g., human leukocyte antigen, HLA) matching, lower incidence of graft-versus-host disease (GvHD), and immediate availability for transplant from storage.

However, because clinical outcomes are driven by cell dose per patient body weight, literature reported 500%-75% of cord blood units collected for US public banking to be discarded, due to having too few cells for clinical use. Of the units banked, only 8% contain enough cells to treat a 150 lb adult at the minimum recommended cell dose, despite adults being the primary recipient of HSC transplants (Bart, Thomas et al. Impact of Selection of Cord Blood Units from the United States and Swiss Registries on the Cost of Banking Operations. Transfus Med Hemother 2013; 40:14-20, Jan. 7, 2013).

One reason for the low yield of collection is the difficulty of cord blood collection. The current standard practice for umbilical cord blood collection requires the doctor performing the delivery to puncture the umbilical vein, a large blood vessel spiraling the length of the umbilical cord, using a large gauge needle connected to a conventional blood bag. After the umbilical vein is punctured, the user is required to hold the collection needle in place, using either a two-hand grip (one for the cord, one for the needle) or a one-hand grip (cord and needle held between different fingers), until all the umbilical and placental blood is gravity-drained. If the bag, line, and needle are not supported, the needle may fall out of the umbilical cord, spilling valuable blood and likely contaminating the sterile sample. This process is time-consuming, requiring several minutes and competes with patient care activities, which the doctor is unable to perform due to the lack of free hands. Due to the simultaneous need to provide patient care, medical staff rarely provide enough time for the cord blood to completely drain. However, the earlier the needle is withdrawn, the lower the volume of blood (and thus cell yield) is collected. Since cord blood is used in Hematopoietic Stem Cell transplant, a minimum number of cells is required (depending on patient body weight). If a cord blood unit does not contain enough cells to be deemed clinically useful, it is discarded, making the collection effort a moot point and a costly waste of materials. Often, staff allow the cord less than one minute to drain, leading to nearly 2.5 times fewer units that meet the minimum cell threshold for banking. Ideally, the collection process would allow the umbilical cord between 5-10 minutes to completely drain. Typical intravenous (IV) catheter stabilization devices/methods (such as taping) or the strategy of advancing long IV cannulas through the umbilical vein are not feasible or reliable in this application due to the umbilical cord's unique helical shape and tissue properties. Even with the help of helping hands or additional time spent collecting blood, the cord blood collection is time-limited by the need to deliver the placenta. Given the flaccid, heavy, and slippery nature of the placenta, it would be difficult and cumbersome for one person to deliver the placenta into a tray (per current practice) while keeping he cord blood collection needle within the vein. having the Having additional persons help in the theoretical maneuver would not significantly diminish the risks of having the cord blood collection needle inadvertently pulling out of the umbilical vein or of having the providers exposed to significant sharps hazards. Thus, cord blood collection remains a time-consuming task that occupies the hands of medical staff.

Therefore, there is a need for a device to secure the collection needle to the umbilical cord vessels.

Additionally, placental perfusion has been explored before as a method of significantly increases cord blood collection yields. Placental perfusion is achieved by creating a sterile and water-tight connection to the vascular system of the umbilical cord and placenta in order to circulate fluid and “wash out” additional cells. The most challenging part of this process is connecting the umbilical arteries to a fluid pump. However, cannulation of umbilical arteries is extremely difficult, as they are small in diameter and tortuous (spiraling inconsistently around the umbilical cord). Current state of the art techniques to access the umbilical arteries for connecting to a perfusion system include methods such as: skilled dissection and re-suturing of vessels onto barbed tube fittings (typically by a vascular surgeon), inserting conventional needles or IV cannulas into the vessel, or cannulating the cut face of the umbilical cord. The first method is impractically labor-intensive; the latter two methods are highly unreliable, because they depend on forcing a length of straight cannula/needle inside a winding, non-linear vessel without damaging or accidentally perforating any of the sidewalls of the vessel, which would lead to extravasation of the infused fluid. However, when the needle is not advanced significantly into the tissue, it is very easy for the cannulae to pull out of tissue when the either tissue or needle moves. This challenge to the access method has prevented the widespread use of placental perfusion as a means of cord blood collection.

Accordingly, it would be beneficial to provide a device for stabilizing and fixing a cannula.

### SUMMARY OF THE INVENTION

The foregoing needs are met, to a great extent, by the present application which, in certain embodiment, provides a device for fixation of a cannula. In one embodiment, the device comprises a hub configured to be coupled to the cannula and a clipping means configured for applying force along the inserted length of the cannula. In another embodiment, the clipping means is further configured to apply force to tissue along the inserted length of the cannula. In another embodiment, the hub surrounds the cannula. In one embodiment, the hub comprises a needle or needle assembly. In one embodiment, the forces applied are onto the tissue along the inserted length of the cannula. In one embodiment, the inserted cannula comprises of one or more cannulae. In one embodiment, the applied force may be applied, sustained, or removed. In another embodiment, the force applied is upon the external of the tissue along the inserted length of the cannula needle. In one embodiment, the clipping means comprises one or more arms which are configured to apply force along the inserted length of the cannula and lock or deform into a position to further secure the device. In one embodiment, the force applied is upon the external of the tissue along the inserted length of the cannula. In one embodiment, the force causes the tissue to fold over the cannula. In one embodiment, the folding of tissue improves the seal of the cannula entrance site into the tissue.

The present application further provides a fixation device for the securement of an intravascular cannula to tissue. In certain embodiments, the fixation device secures the intravascular cannula to tissue by applying force on the exterior of the tissue along the inserted length of the cannula, or on the exterior of the tissue just prior to the point of insertion. In other embodiments, the force comprises compression, friction, interference, adhesives, or magnetism. In one embodiment, the position of the inserted cannula serves as a means of accurately placing the fixation features. In one embodiment, the inserted length of cannula comprises one or more cannulae. In one embodiment, the force causes the tissue to fold over the cannula. In one embodiment, the folding of tissue improves the seal of the cannula entrance site into the tissue.

The present application also provides a method for fixing an intravascular cannula to tissue. In one embodiment, the method comprises applying force on the exterior of the tissue, along the inserted length of the cannula or just prior to the point of insertion. In one embodiment, the position of the cannula is utilized as a reference for accurate placement of the fixation features. In one embodiment, the inserted length of cannula comprises one or more cannulae. In one embodiment, the force causes the tissue to fold over the cannula. In one embodiment, the folding of tissue improves the seal of the cannula entrance site into the tissue.

The present application provides a placental perfusion device. In one embodiment, the placental perfusion device consists of two containers and an assembly or object for storing the potential energy required for driving the perfusate from a first container through the placenta into a second container. In one embodiment, the first and second containers are selected from fluid bags, deformable containers, or syringes. In one embodiment, the assembly or object stores potential energy by means of a compressed spring or an elastic element. In another embodiment, the assembly or object stores potential energy by means of physical elastic deformation of an element of the assembly or object. In one embodiment, the deformed element of the assembly or object is the assembly or object's frame. In one embodiment, the assembly or object stores potential energy by means of a battery. In another embodiment, the assembly or object stores potential energy by means of air pressure. In one embodiment, the entirety of energy required for a perfusion process is deposited by a user and utilized throughout a perfusion process without the need for further user interaction.

The present application further provides a method of placental perfusion. In one embodiment, the method utilizes stored energy in an assembly or object to drive fluid from a first container through the placenta into a second container. In one embodiment, the first and second containers are selected from fluid bags, deformable containers, or syringes. In one embodiment, the assembly or object stores potential energy by means of a compressed spring or an elastic element. In another embodiment, the assembly or object stores potential energy by means of physical elastic deformation of an element of the assembly or object. In one embodiment, the deformed element of the assembly or object is the frame of the assembly or object. In one embodiment, the assembly or object stores potential energy by means of a battery. In another embodiment, the assembly or object stores potential energy by means of air pressure. In one embodiment, the entirety of energy required for a perfusion process is deposited by a user and utilized throughout a perfusion process without the need for further user interaction.

### BRIEF DESCRIPTION OF THE DRAWINGS

FIG. 1 is a perspective view of an embodiment of the intravascular fixation device (with cannula) in an open position.

FIG. 2 is a perspective view of the device (with cannula) of FIG. 1 in a closed position.

FIG. 3 is a perspective view of the device of FIG. 1 without the cannula (1) present.

FIG. 4 is a close-up perspective view of the device (with cannula) of FIG. 1 in an open position.

FIG. 5 is a front view of the device (with cannula) of FIG. 1 in an open position.

FIG. 6 is a perspective view of an alternative embodiment of the intravascular fixation device (with cannula) in an open position.

FIG. 7 is a perspective view of the device of FIG. 6 (with cannula) in a closed position.

FIG. 8 is a perspective view of the device of FIG. 6 without the cannula (13) present.

FIG. 9 is a close-up perspective view of the device (with cannula) of FIG. 6 in an open position.

FIG. 10 is a front view of the device of FIG. 6 (without cannula) in an open position.

FIG. 11 is a perspective view of an embodiment of the intravascular fixation device (with cannula) in an open position.

FIG. 12 is a close-up perspective view of the device of FIG. 11 (with cannula) in an open position.

FIG. 13 is a front view of the device of FIG. 11 (with cannula) in an open position.

FIG. 14 is a perspective view of the device of FIG. 11 without the cannula present.

FIG. 15 is a top view of the device of FIG. 11 (with cannula) in an open position.

FIG. 16 is a perspective view of an embodiment of the intravascular fixation device (without cannula) in an open position.

FIG. 17 is a top view of the device of FIG. 16 without cannula present.

FIG. 18 is a front view of the device of FIG. 16 without cannula present.

FIG. 19 is a perspective view of the backside of device of FIG. 16 without cannula present.

FIG. 20 is a side view of the device of FIG. 16 without cannula present.

FIG. 21 is a perspective view of the backside of the device of FIG. 16 without cannula present.

FIG. 22 is a perspective view of the bottom side of the device of FIG. 16 with cannula partially inserted.

FIG. 23 is a perspective view of the bottom side of the device of FIG. 16 with cannula inserted but prior to pivoting to lock cannula in place.

FIG. 24 is a perspective view of the device of FIG. 16 with cannula fully inserted.

FIG. 25 is a perspective view of the device of FIG. 16 with cannula inserted and cannula cap removed.

FIG. 26 is a top view of the device of FIG. 16, with uncapped cannula, in a closed configuration.

FIG. 27 is a section view of side of device of FIG. 16, with cannula inserted into a vessel in tissue.

FIG. 28 is a top view of the device of FIG. 16 in closed configuration, secured onto cannulated tissue.

FIG. 29 is an open view of the closed bag compressor.

FIG. 30 is an open view of the bag compressor.

FIG. 31 is a cross-sectional view of the bag compressor compressing with infusion bag.

### DETAILED DESCRIPTION

The presently disclosed subject matter now will be described more fully hereinafter with reference to the accompanying Drawings, in which some but not all embodiments of the invention are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention is directed to a device for fixation, stabilization, and securement of an intravascular cannula to tissue. The fixation device affixes relative to the cannula and cannulated tissue, allowing the needle to be inserted and then rapidly secured with one motion, using just one hand. The device works to secure cannulae not only in small vessels but also in situations non-permissive to current state-of-the-art cannulas—such as on wet, adhesive-incompatible surfaces, in intra-operative applications, or when hands-free securement is required. Furthermore, the device allows for very shallow penetration depths by negating the risk of cannula pull-out from tissue upon inadvertent movement of the tissue or cannula. The device can be integrated with a cannula (e.g., IV needle) or used add-on component. The device of the present invention is relevant to phlebotomy or tissue perfusion applications, but can also be used in any other suitable application known to or conceivable by one of skill in the art.

As used in this description and the accompanying claims, the following terms shall have the meanings indicated below, unless otherwise stated in context.

A “Cannula” shall mean any narrow, hollow instrument capable of transferring fluids through it; for example, but not limited to, a needle or catheter.

The term “clipping means” is used herein to refer to any physical feature or components for the securement of the device unto the tissue; for example, but not limited to, moving arms or surfaces, barbs or hooks, or surfaces that grab or press against the tissue, or dispensing of adhesives. “Clip” is utilized to refer to an embodiment of a clipping means.

One preferred embodiment of the cannula stabilization device assembly, shown in FIG. 1-5, includes a conventional needle cannula (shown with a Luer lock base) 1 and hub 2, affixed to the body of the cannula needle. The hub includes of two parallel arms to either side of the cannula 3, attached to the cannula only through the body of the hub and affixed proximal to the tip of the cannula. Along the arm, there is a concave section to the arm that serves to provide an ergonomic handle to allow users to easily grip and handle the assembly with their fingertips 4. The clamping sections are located at the end of each arm 5. Each arms' clamping section are shaped to pinch and grip the tissue area underneath the cannula. This causes the tissue to “fold” over the cannula, with its tension helping to maintain the seal of the puncture site. Each clamping section also has a claw or hook-like protrusions that enhance securement onto soft tissue. These clamping sections start in the open and un-joined position to allow tissue to slide past the cannula tip and gripping sections as shown in FIG. 1.

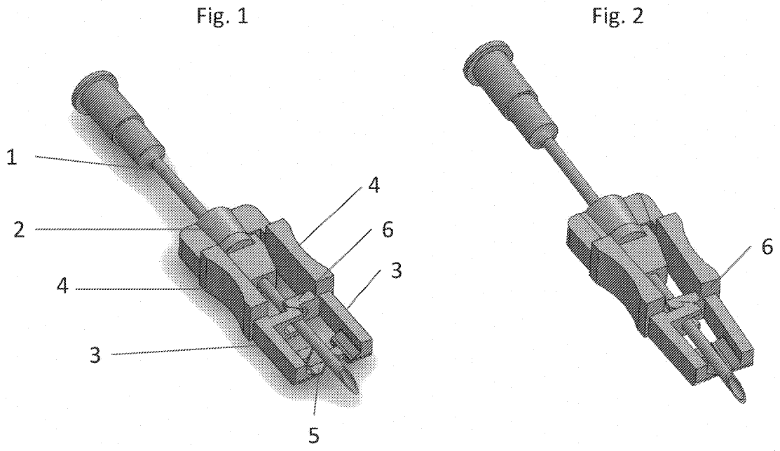

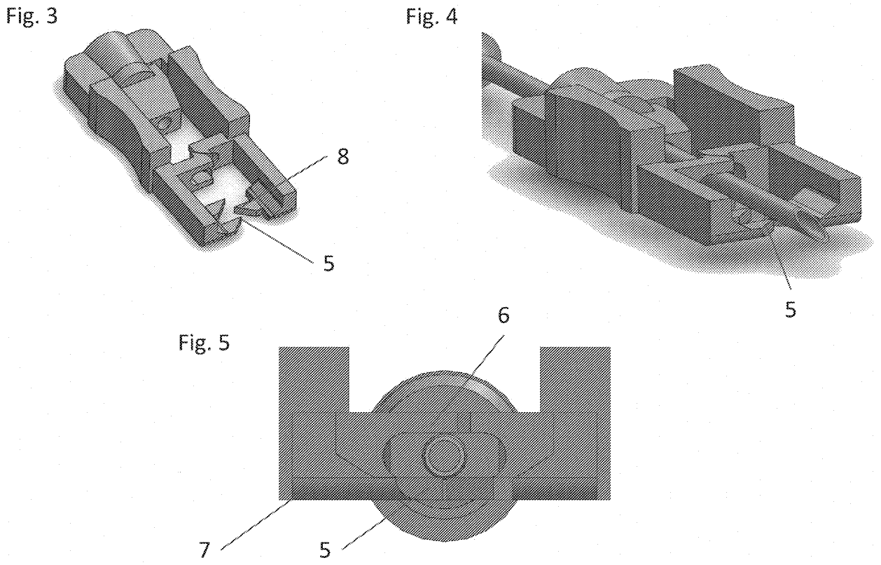

Once the cannula has punctured the tissue to a minimum depth, as indicated by an identified FIG. 3, Feature 8 and ready to be locked onto the tissue, the user simply squeezes close the arms by applying inward force on both ergonomic grip sections, similar to a “tweezer forceps”, causing the clamping sections to close and secure onto the tissue within the clamping sections (shown in FIG. 2), securing and locking the spatial relationship between the tissue and the cannula-tip. There is a locking mechanism 6 that includes two mirrored and opposite snap fits and is located on either arm at the opposite face of the ergonomic handle section. These snap fits secure together to hold the clamping sections in the closed position. The flexibility of the arms provides steady and constant pinching pressure and are designed with a rounded tip 7 to allow soft tissue to slide past easily during insertion. In alternative embodiments, the parallel arms may start non-parallel or bend inwards to grasp the tissue from any conceivable angle or direction.

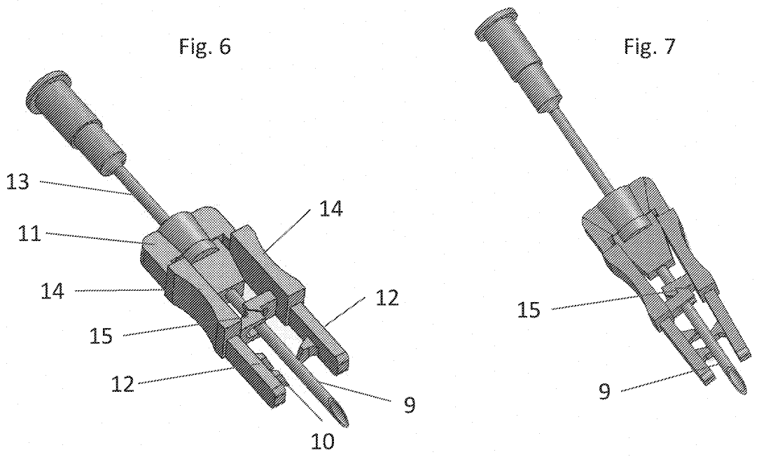

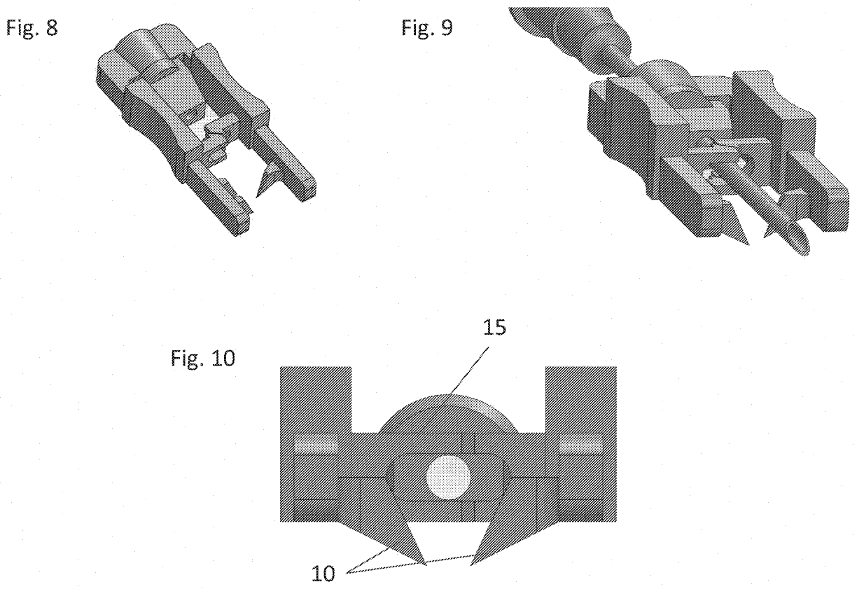

An alternative embodiment of the stabilization assembly is shown in FIGS. 6-10. Instead of the clamping section interfacing with the tissue in the region along the inserted length of the cannula, clamping features are located and interact with the section of tissue proximal to the puncture site (9 shows the depth at which the cannula is inserted into the tissue, indicating the position of the tissue surface upon the cannula). After insertion, the arms are actuated such that the claw-like features 10 pinch and secure onto the tissue proximal to the puncture site. Once secured onto with the tissue, this locks the special relationship between the cannula and the tissue due to the proximity to the insertion site.

Similar to the preferred embodiment, the clipping means consists of the hub 11 and its corresponding arms 12, which are affixed to the cannula 13. The cannula is inserted into the tissue until a point such as 9, such that the tissue-grasping features 10 are as close as possible, but not distal to the point of insertion. The user squeezes the two arms together using the ergonomic pads 14, and the interlocking clasps 15 hold the two arms in the closed position, as shown in FIG. 7. In this motion, the tissue-grasping features 10 dig into and pinch the tissue near the point of insertion to secure the hub and cannula to the tissue.

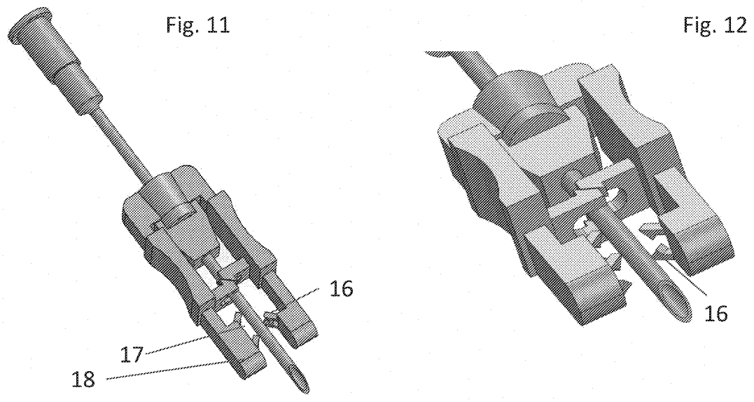

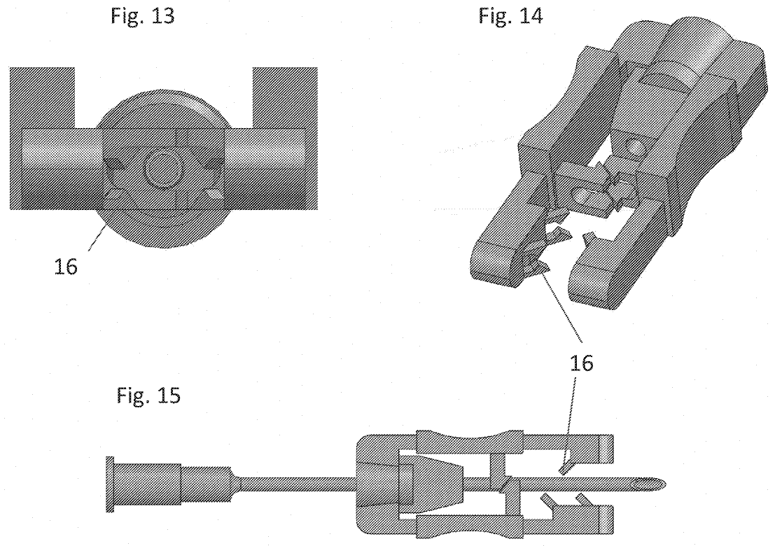

Another embodiment is shown in FIGS. 11-15. Similar to the previously described embodiments, this embodiment has a series of directionally biased barb features 16 in the clasping sections 17 of its arms 18 which function to slide over the tissue upon insertion of the cannula and prevent the cannula from sliding back out of the tissue. The barbs may displace in contact with the tissue, such as while sliding over tissue, or the arms may be closed (as described in other embodiments) to secure the barbs onto the tissue.

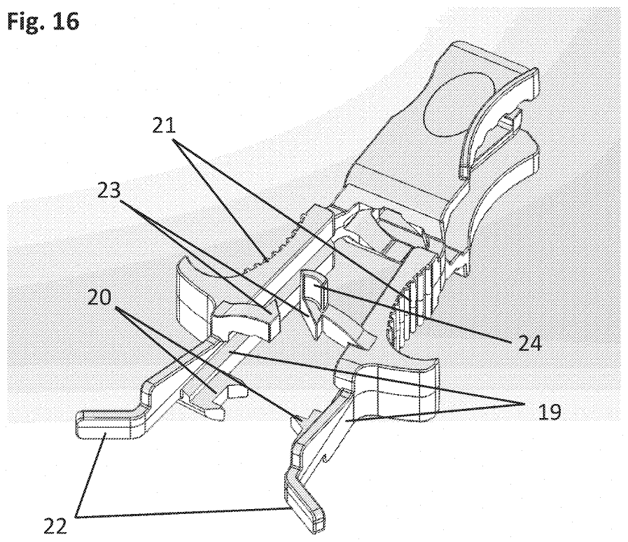

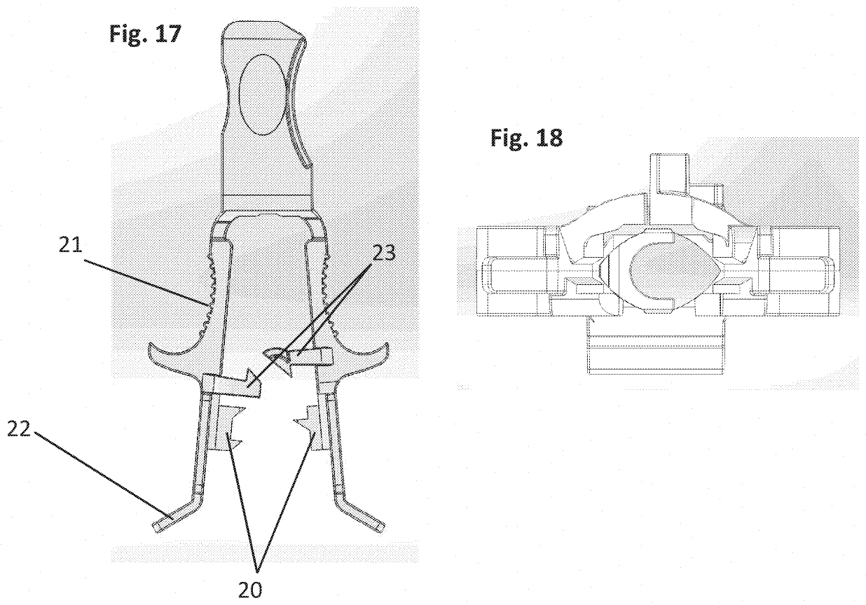

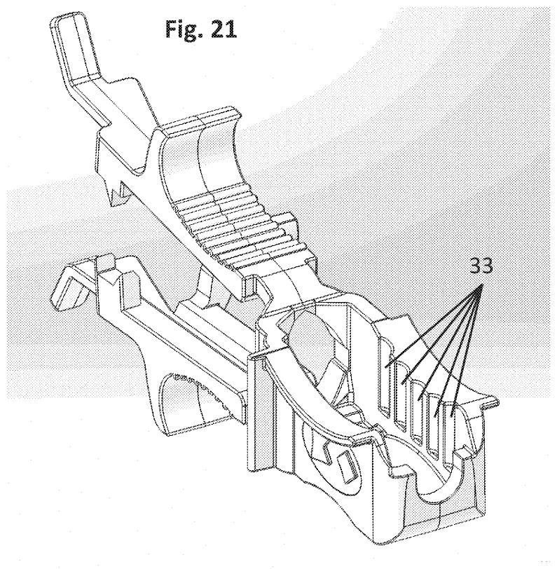

Another embodiment, intended to be coupled with a needle assembly (needle with in-built hub) at point of use by user, is shown in FIGS. 16-28. The needle assembly shown is a common cord blood collection bag's needle.

FIG. 16 shows the perspective view of this embodiment. Like previous embodiments, it has similar features such as two flexibly deforming arms 19, tissue-clasping sections with barb-like features 20, and finger-gripping areas 21 to facilitate holding the arms with index finger and thumb. This embodiment further incorporates guard-arms 22 to protect stray fingers manipulating cannulated tissue from being accidentally punctured by the needle after attached to tissue. The embodiment's central latching mechanism 23 also incorporates a protruding feature 24 for ease of unlatching the latching mechanism 23 and opening the device, should repositioning of the cannula be needed.

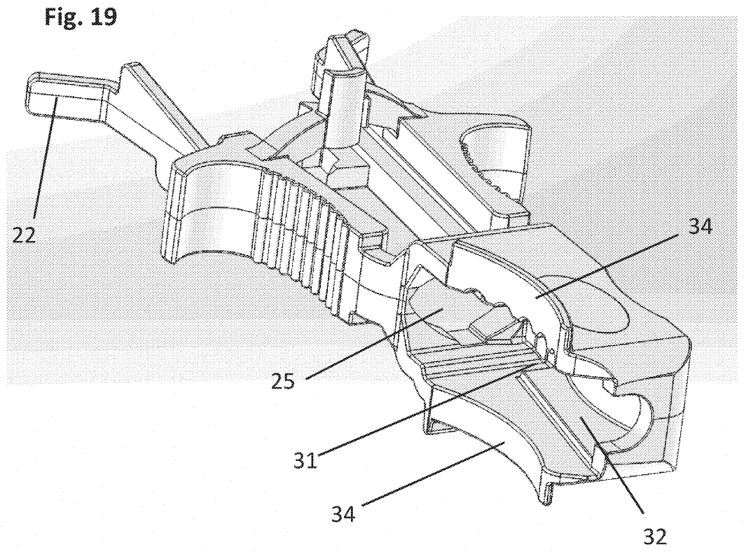

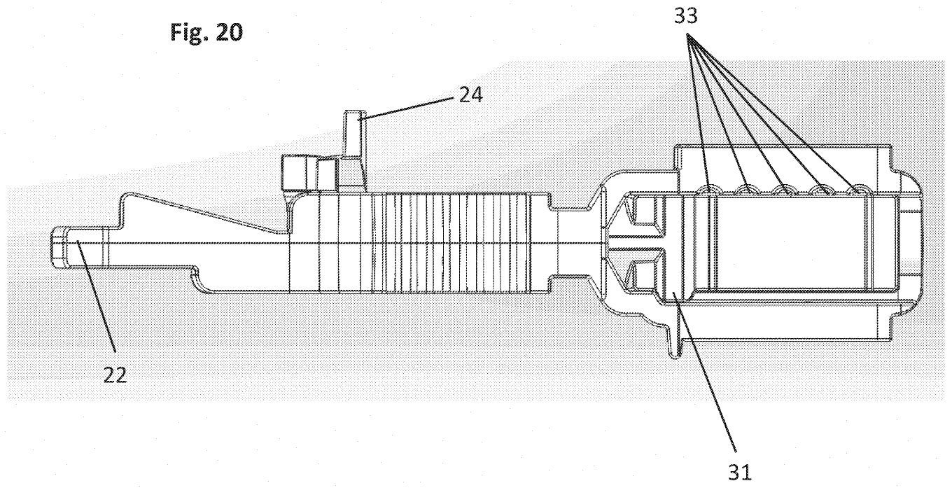

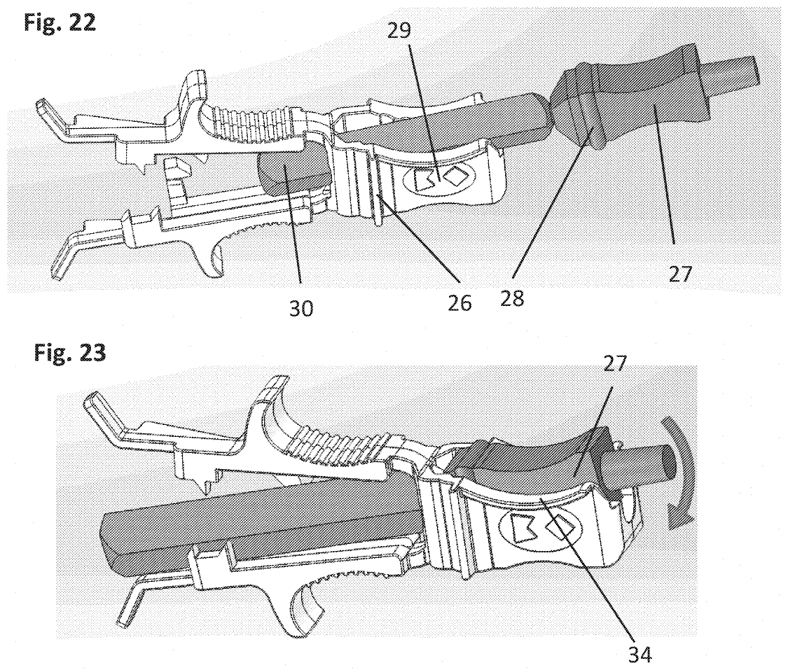

FIG. 19 and FIG. 20 show the perspective view of the device's backside and side view, respectively. The conventional cord blood bag's needle and cap (not shown) are inserted point-first through the device's central hole 25, as shown in FIG. 22.

As seen in FIG. 22, an orientation feature 26 is present and correlates to the position of a needle feature, such as the needle's hub's 27 bump 28. This is used to correctly orient the needle for insertion into the device “bevel down”. Other features, such as symbols or graphics 29 corresponding to other needle features may be utilized as well. The needle cap 30 is not removed until after device attachment.

FIG. 19 shows a groove 31 for the needle hub's bump 28 to fit into by interference fit. The device's body is shaped in areas 32 to allow the needle to be inserted at an angle and pivoted into a locked position, wherein it is supported in the direction that resistance is expected (as shown in FIGS. 22 and 23). FIG. 20 shows a further feature 33 designed to “catch” the needle hub's bump 28 to make it difficult to incorrectly insert the needle assembly in a “bevel-up” orientation. Flanges 34 are also present to provide a point of leverage for removal of needle assembly from device.

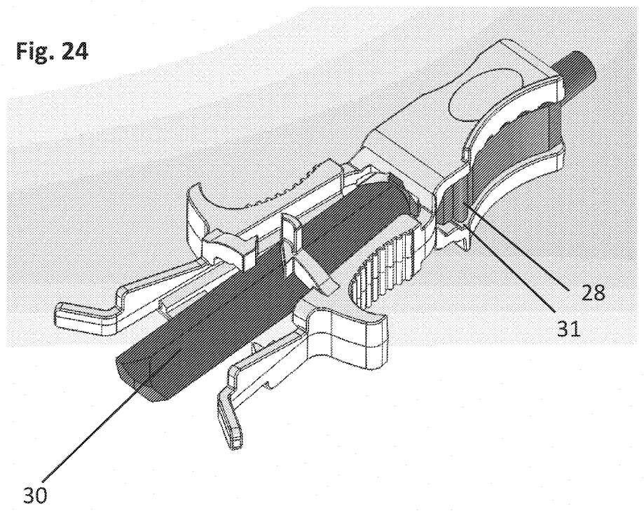

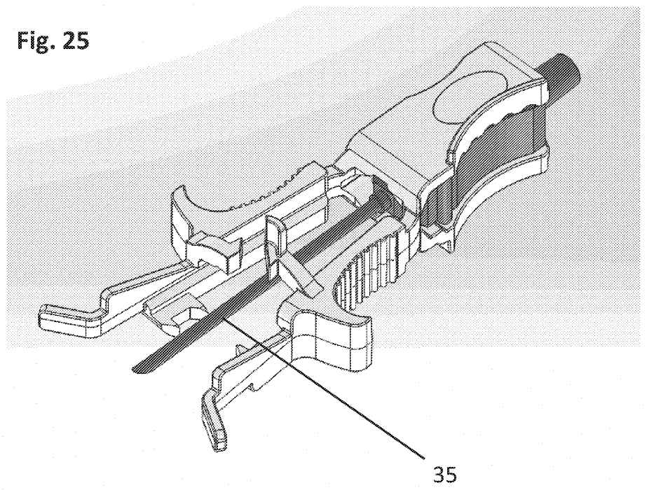

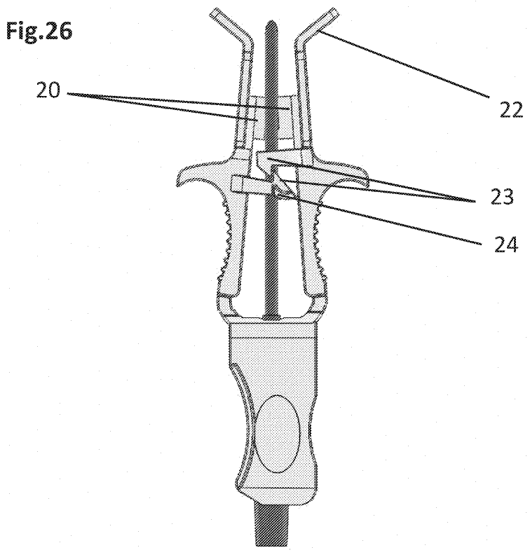

Following insertion of needle assembly into device (FIG. 22), pivoting to secure (FIG. 23), and removal of needle cap 30 (FIG. 24-25) to expose the needle 35, the device may be closed to engage any tissue the cannula may be placed into. In this state, the central latching mechanism 23 (FIG. 26) holds the device closed until released by user.

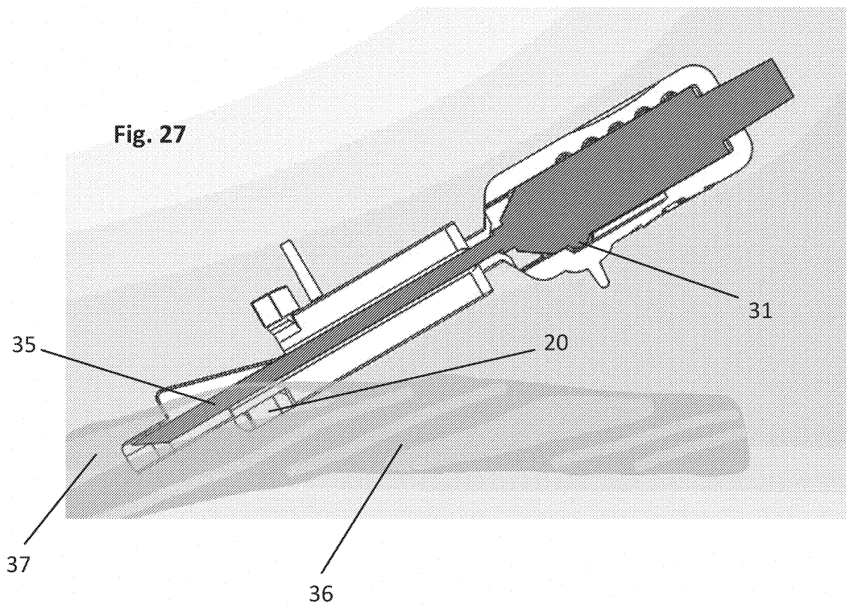

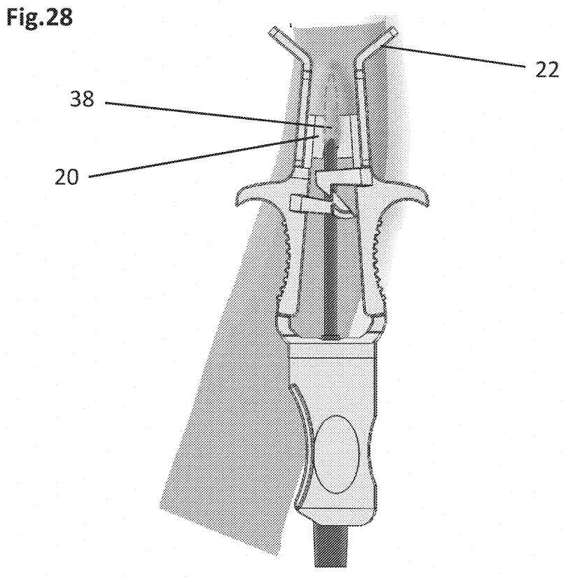

As shown in FIG. 27-28, with device open, tissue 36 is cannulated, placing the needle 35 within a vessel 37 deeply enough for the clasping sections 20 to engage the tissue 36 when the device is closed. When closed, the clasping sections 20 engage the tissue, not only securing onto the tissue but also “wrapping” the tissue around the needle 38. Guard-arms 22 prevent accidental needle stick in further manipulation of the tissue by user.

The cannula fixation device described herein features a novel method of securement. The device affixes to the exterior of the tissue of the blood vessel along the inserted length of the cannula (or just proximal to its entrance into the tissue) by means of fixation features that utilize forces such as interference, friction, or adhesives. In one embodiment, the fixation features are positioned accurately by utilizing the cannula as a point of spacial reference.

Such an arrangement allows for a very short puncture distance by minimizing the distance in location between the cannula's tip and the mechanical anchor point on the tissue. This minimizing this distance reduces the displacement of the needle tip during pivoting motions around the anchor point during the inevitable agitation of the cord, needle, bag, and tubing during use. As such, the stability this anchoring method and location produces at the tip of the needle prevents all undesirable motions: needle pull-out, needle advancing too far and piercing through back wall of vessel, needle pivoting/angling and cutting the walls of vessel. Part of the robustness of its stability also comes from fixation's ability (to a limited degree) to move/pivot the elastic vessel wall to keep the same desired spatial orientation with respect to the needle tip.

Once anchored, the task of draining the umbilical cord for conventional cord blood collection can be a hands-free task. This further allows the collection bag to stay connected through and after the delivery of the placenta, extending the total time of cord blood collection. The user anchors the cannula to the umbilical cord and lets the cord, cannula, and collection bag tubing rest or hang freely. Over time, gravity will allow the cord blood to flow into the collection bag. Alternatively, the now unoccupied hands of the user may now be utilized for the technique of ‘milking’ the umbilical cord towards the collection bag, further increasing both collection volume and speed.

Furthermore, this device enables fast placental perfusion setup. The stability relative to the puncture site allows for extremely shallow penetration depths, (e.g., 3 mm of insertion), where previously, even tissue stretching or introduction of fluid would threaten catastrophic needle-pull-out Instead, the inventors are able cannulate small and tortuous vessels (umbilical cord vessels spiral and are difficult to visualize) without risk of puncturing through the back wall, because deep insertion is not required. Additionally, the locking component (discussed in herein) assists in sealing the puncture site, such that any positive pressure of introducing fluids (e.g., perfusion) faces minimal risk of leakage. As such, this device negates the obstacles associated with access to the umbilical arteries for purposes of placental perfusion. The device's simplicity, speed, and reliability enable the practical use of placental perfusion for cord blood collection in the medical setting.

The cannula fixation invention also presents an improvement in ease-of-use for short-term needle stabilization applications (e.g., cord blood collection, routine blood draw or Umbilical Blood Gas procedure, using a BD vacutainer system). If the cannula fixation device was integrated with a needle, it would allow allowing the phlebotomist to simply poke, then “lock” the needle in place in one action, using one hand, without any additional items or steps required. Additionally, the invention can be utilized in situations non-permissive to conventional securing means, such as non-dry or greasy surfaces, small surface areas, or intra-operatively (vessels in the body). The cannula fixation device provides a fixation method which can be used to provide fast and robust intravascular cannula stabilization and fixation to the cannulated tissue subsequent to the insertion of a cannula. Another objective of the invention to provide the device with an ergonomic grip section that conforms to user's natural in a griping position and motion. Applying a certain pressure on the grip leads to the device's switching between positions; going from its open position, used during insertion of the cannula, to its closed position, used to fixate the cannula to the tissue.

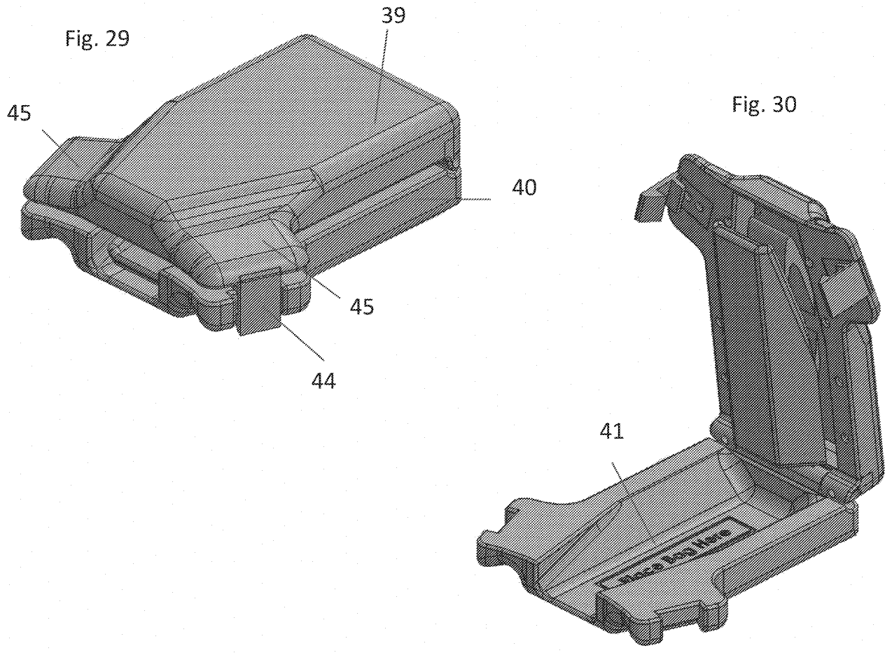

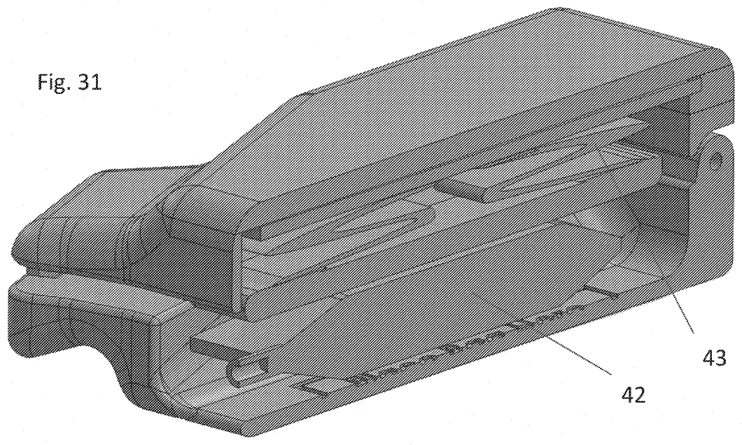

FIGS. 29-31 shows a preferred embodiment of a bag compression device, utilized in placental perfusion for collection of cord blood. The compressor consists of two halves 39 and 40 that can open and close as shown in FIGS. 29 and 30. When opened, an infusion bag containing perfusate solution may be placed inside a receiving cavity 41. When the compressor is again closed (FIG. 31), pressure is applied to the infusion bag 42 by means of a compressed spring 43. The two halves of the compressor, 39 and 40, are held closed by latches 44.

The bag compression device allows for a simple method to drive a perfusion system wherein the perfusate is contained within a pressurizable or deformable container (bag, syringe, etc.). Additionally, the compression of the spring (or other method of loading the system, such as elastic members or pressurization) is a fast means of depositing the entirety of energy required for the perfusion process within a single, short interaction with the user. A motorized method of loading or deforming the container may also be used. After the compression of the spring, no additional user interaction is needed for the perfusion process. Furthermore, the compression device is designed with ergonomic handles 45 such that, when placed on a flat surface, allow the user to press downwards with a large portion of their bodyweight in a simple gross motor movement. The use of bodyweight to load energy allows even users lacking hand or arm strength to be able to load the device with a great amount of energy that may be required to drive the perfusion process. As the device controls the perfusion process, the possibility of user variation is removed, ensuring a more reliable and reproducible placental perfusion cord blood collection process.

The many features and advantages of the invention are apparent from the detailed specification, and thus, it is intended by the appended claims to cover all such features and advantages of the invention, which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device for fixation of a cannula comprising:
a hub configured to be coupled to the cannula; and
a clipping means configured for applying force along the inserted length of the cannula.

2. The device of claim 1, wherein the clipping means is further configured to apply force to tissue along the inserted length of the cannula, and wherein the applied force may be applied, sustained, or removed.

3-5. (canceled)

6. The device of claim 1, wherein the inserted cannula comprises one or more cannulae.

7. (canceled)

8. The device of claim 2, wherein the force applied is upon the external of the tissue along the inserted length of the cannula needle.

9. The device of claim 1, wherein the clipping means comprises one or more arms which are configured to apply force along the inserted length of the cannula and lock or deform into a position to further secure the device.

10. The device of claim 9, wherein the force applied is upon the external of the tissue along the inserted length of the cannula.

11. The device of claim 9, wherein the force causes the tissue to fold over the cannula.

12. The device of claim 11, wherein the folding of tissue improves the seal of the cannula entrance site into the tissue.

13. A fixation device for the securement of an intravascular cannula to tissue by applying force on the exterior of the tissue along the inserted length of the cannula, or on the exterior of the tissue just prior to the point of insertion, wherein the force comprises compression, friction, interference, adhesives, or magnetism.

14. The device of claim 13, wherein the position of the inserted cannula serves as a means of accurately placing the fixation features.

15. The device of claim 13, wherein the inserted length of cannula comprises one or more cannulae.

16. The device of claim 13, wherein the force causes the tissue to fold over the cannula.

17. (canceled)

18. A method for fixing an intravascular cannula to tissue using the fixation device of claim 13.

19-20. (canceled)

21. The method of claim 18, wherein the force causes the tissue to fold over the cannula, and wherein the folding of tissue improves the seal of the cannula entrance site into the tissue.

22. (canceled)

23. A placental perfusion device consisting of two containers and an assembly or object for storing the potential energy required for driving the perfusate from a first container through the placenta into a second container.

24. The device of claim 23, wherein the first and second containers are selected from fluid bags, deformable containers, or syringes.

25. (canceled)

26. The device of claim 23, wherein the assembly or object stores potential energy by means of physical elastic deformation of an element of the assembly or object.

27. The device of claim 26, wherein the deformed element of the assembly or object is the assembly or object's frame.

28. (canceled)

29. The device of claim 23, wherein the assembly or object stores potential energy by means of air pressure.

30. (canceled)

31. A method of performing placental perfusion using the placental perfusion device of claim 23.

32-38. (canceled)
